(19) United States
(12) Patent Application Publication
(10) Pub. No.: US 2018/0117239 A1
(43) Pub. Date: May 3, 2018

(54) Interface Tubing for Peristaltic Pump

(71) Applicant: St. Jude Medical, Cardiology Division, Inc., St. Paul, MN (US)

(72) Inventors: Brian Schmidt, Cottage Grove, MN (US); Coleman Leach, St. Paul, MN (US); Michael Kloosterboer, Minneapolis, MN (US); Lawrence D. Swanson, White Bear Lake, MN (US); Edward E. Parsonage, St. Paul, MN (US)

(21) Appl. No.: 15/727,847

(22) Filed: Oct. 9, 2017

Related U.S. Application Data
(60) Provisional application No. 62/416,204, filed on Nov. 2, 2016.

(51) Int. Cl. A61M 3/02; A61B 5/042; A61L 29/08; A61L 29/14; B29C 47/00; B29C 47/06; B05D 1/02; B05D 1/18; B29C 63/00

(52) U.S. Cl. CPC A61M 3/0279; B29L 2031/7542; A61M 3/0258; A61L 29/08; A61L 29/14; B29C 47/0023; B29C 47/065; B05D 1/02; B05D 1/18; B29C 63/0069; A61M 2207/00; A61M 2205/0238; A61M 2205/42; A61M 2205/0233; A61L 2420/02; A61L 2420/08; A61B 5/0422

(57) ABSTRACT

A pump interface tubing for use in a peristaltic pump includes a tubular core having an outer surface and a treatment on the outer surface. The treatment reduces static charge buildup on the tubular core during operation of the peristaltic pump, and thereby reduces the noise signal that might otherwise undesirably couple to a signal of interest. Treatments include nitrile layers, heat shrink layers, cotton fiber layers, and anti-static sprays.

### CROSS-REFERENCE TO RELATED APPLICATIONS

This application claims the benefit of U.S. provisional application No. 62/416,204, filed 2 Nov. 2016, which is hereby incorporated by reference as though fully set forth herein.

### BACKGROUND

The instant disclosure relates to irrigated electrophysiology catheters. In particular, the present disclosure relates to devices and methods for reducing electrical noise in an irrigated electrophysiology catheter system.

Catheters are used for an ever growing number of medical procedures. To name just a few examples, catheters are used for diagnostic, therapeutic, and ablative procedures. Typically, the physician manipulates the catheter through the patient's vasculature to the intended site, such as a site within the patient's heart. The catheter typically carries one or more electrodes (in the case of so-called “electrophysiology catheters”) or other diagnostic or therapeutic devices, which can be used for ablation, diagnosis, cardiac mapping, or the like.

Irrigated electrophysiology catheters are also known. An irrigated electrophysiology catheter is an electrophysiology catheter that is equipped to deliver an irrigation fluid, such as saline, to a location proximate the electrodes. The irrigation fluid serves, for example, to cool the electrodes or to disperse body fluids therefrom, to cool or bathe surrounding tissue, and/or to couple the electrodes to the tissue surface in the case of relatively highly conductive fluid(s).

In many irrigated electrophysiology catheters, a peristaltic pump is used to deliver the irrigation fluid. Typical peristaltic pumps operate by rotating a number of rollers mounted on a rotor to periodically compress an irrigation tube between the rollers and a pump housing or clamp, which forces the irrigation fluid through the irrigation tube.

It is known, however, that peristaltic pumps may generate electrical noise that can undesirably couple to an electrogram signal measured by the electrodes on the irrigated electrophysiology catheter, to an electroanatomical visualization, localization and/or position system coupled to a subject, and/or to other diagnostic or therapeutic equipment. This noise is referred to herein as the “noise signal.” The components of the noise signal include electrostatic discharge (“ESD”).

### BRIEF SUMMARY

Disclosed herein is a pump interface tubing for use in a peristaltic pump that includes: a tubular core having an outer surface; and a treatment on the outer surface of the tubular core, wherein the treatment reduces static charge buildup on the tubular core during operation of the peristaltic pump. The tubular core can include a first material and the treatment can include a layer of a second material different from the first material formed about the tubular core. Alternatively, the treatment can include a layer of the first material mixed with an ESD-preventing additive formed about the tubular core. The treatment can extend along any or all of the length of the outer surface of the tubular core.

It is contemplated that the second material will be closer to zero on the triboelectric scale than the first material. For example, the second material can be a nitrile, a heat shrink material, or a cotton fiber. Alternatively or additionally, the treatment can include an anti-static spray treatment.

In embodiments of the disclosure, the treatment can also resist abrasion of the tubular core during operation of the peristaltic pump.

The pump interface tubing can also include first and second keys at opposite ends thereof. The keys can be used to interconnect the pump interface tubing to an irrigation tube.

Also disclosed herein is an irrigation system for use with an irrigated electrophysiology catheter, including: a peristaltic pump; and a pump interface tubing. The pump interface tubing includes: a tubular core having an outer surface; and a treatment on the outer surface of the tubular core, wherein the treatment reduces a noise signal resulting from operation of the peristaltic pump.

In embodiments of the disclosure, the treatment reduces static buildup on the pump interface tubing resulting from operation of the peristaltic pump. For example, the treatment can include an anti-static layer (e.g., a nitrile layer, a heat shrink layer, a cotton fiber layer, and/or a layer of an anti-static spray) about the tubular core.

According to another aspect of the disclosure, a method of manufacturing a reduced-noise pump interface tubing for use in a peristaltic pump, includes forming a noise-reduction layer about a tubular core.

The noise-reduction layer can be formed by forming a nitrile layer about the tubular core, such as by dip-coating the nitrile layer about the tubular core.

The noise-reduction layer can also be formed by co-extruding the tubular core from a first material and the noise-reduction from a second material different from the first material.

In still other embodiments, the noise-reduction layer can be formed by spraying the tubular core with an anti-static spray.

In further embodiments, the noise-reduction layer can be formed by forming a cotton fiber layer about the tubular core.

In still further embodiments, the noise-reduction layer can be formed by forming a heat shrink layer about the tubular core.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 depicts a peristaltic pump.

FIG. 2 is a representative electrostatic discharge noise signal.

FIG. 3 is an illustrative transverse cross-section of a pump interface tubing that depicts the tubular core and the noise reduction layer disclosed herein.

FIG. 4 is a representative bipolar electrogram according to aspects of the instant disclosure that utilize a nitrile noise reduction layer.

FIG. 5 is a representative bipolar electrogram according to aspects of the instant disclosure that utilize a cotton fiber noise reduction layer.

FIG. 6 is a representative bipolar electrogram according to aspects of the instant disclosure that utilize an acrylated olefin heat shrink noise reduction layer.

FIG. 7 is a representative bipolar electrogram according to aspects of the instant disclosure that utilize a co-extruded non-DEHP noise reduction layer.

FIG. 8A is a representative bipolar electrogram according to aspects of the instant disclosure that utilize a general purpose staticide noise reduction layer.

FIG. 8B is a representative bipolar electrogram according to aspects of the instant disclosure that utilize a licron crystal staticide noise reduction layer.

### DETAILED DESCRIPTION

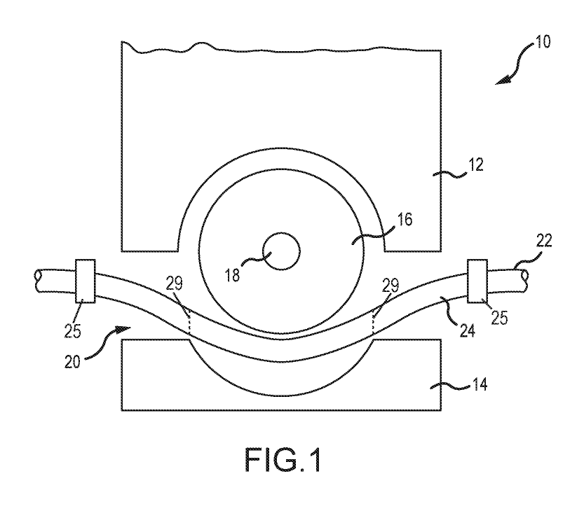

FIG. 1 depicts a peristaltic pump 10, such as the Cool Point™ Irrigation Pump of St. Jude Medical, Inc. The configuration and operation of peristaltic pump 10 will be familiar to those of ordinary skill in the art (see, e.g., United States patent application publication no. 2007/0224063, which is hereby incorporated by reference as though fully set forth herein), such that a detailed explanation thereof is not necessary herein. Instead, only those features of peristaltic pump 10 pertinent to understanding the present disclosure will be described below.

Peristaltic pump 10 generally includes a housing 12, a clamp 14, and a rotor 16. Rotor 16 includes a plurality of rollers spaced about the circumference of rotor 16 and is mounted to rotate about an axle 18.

A tubing channel 20 is defined between clamp 14 and rotor 16. Tubing channel 20 accommodates an irrigation tube 22. One end of irrigation tube 22 can be coupled to a suitable reservoir of irrigation fluid, while the opposite end of irrigation tube 22 can be coupled to an irrigated electrophysiology catheter. Thus, when in operation, peristaltic pump 10 moves irrigation fluid from the reservoir into the electrophysiology catheter, where it moves through one or more irrigation lumens and exits via one or more irrigation ports.

A portion of irrigation tube 22, referred to herein as the pump interface tubing 24, is positioned between clamp 14 and rotor 16, and is interconnected to the remainder of irrigation tube 22 by keys 25. One of ordinary skill in the art will appreciate that, as rotor 16 turns, the rollers will periodically (if evenly spaced about the circumference of rotor 16) impinge upon pump interface tubing 24, pushing pump interface tubing 24 against clamp 14 and forcing fluid through irrigation tube 22 to provide a pulsatile flow of irrigation fluid to the electrophysiology catheter.

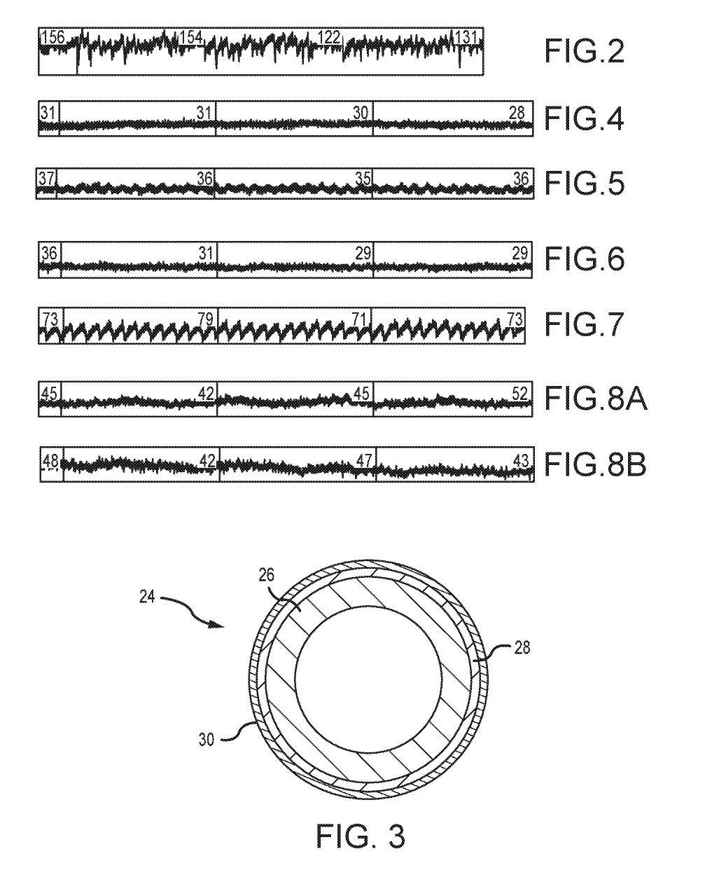

As described above, operation of peristaltic pump 10 can create a noise signal due, inter alia, to ESD. The noise signal can undesirably couple to an electrical signal of interest (e.g., a bipolar electrogram measured using intracardiac electrodes). FIG. 2 shows an illustrative ESD noise signal.

The instant disclosure provides various modifications and improvements to pump interface tubing 24 that desirably reduce or eliminate the noise signal. Several embodiments of the disclosure are illustrated schematically in FIG. 3, which is a transverse cross-section of pump interface tubing 24 according to aspects disclosed herein.

As shown in FIG. 3, pump interface tubing 24 includes a flexible, polymeric, and tubular core 26. Tubular core 26 can be made of a non-DEHP tubing, such as Tygon® ND-100-65 from Saint-Gobain Performance Plastics, which uses TOTM as a plasticizer. The use of non-DEHP materials for tubular core 26 is desirable in that it preserves biocompatibility, though other biocompatible materials can be used for tubular core 26 without departing from the scope of the instant teachings.

FIG. 3 also shows a treatment 28 on the outer surface of tubular core 26. As discussed in greater detail below, treatment 28 reduces static charge buildup on tubular core 26 during operation of peristaltic pump 10, and thus reduces the noise signal resulting from operation of peristaltic pump 10. Thus, treatment 28 is also referred to herein as a “noise-reduction layer.”

As shown in FIG. 3, it is desirable for treatment 28 to cover the complete circumference of tubular core 26. It is also desirable for treatment 28 to be sufficiently thick (e.g., in the radial direction in FIG. 3) that it is not compromised during operation of peristaltic pump 10 in a manner that exposes tubular core 26 to rotor 16. In this regard, in embodiments of the disclosure, an additional layer 30 of protective material can be applied over treatment 28 in order to enhance the durability of treatment 28. Layer 30 can include, by way of example only, wax, zinc stearate, erucamide, and other abrasion-resistant materials. The material used in layer 30 can be selected so as not to impair the ability of treatment 28 to reduce static charge buildup on tubular core 26 during operation of peristaltic pump 10.

In embodiments of the disclosure, treatment 28 is applied to the entire length of pump interface tubing 24 (e.g., the entire length between keys 25 in FIG. 1). In other embodiments, treatment 28 is applied only to the portion of pump interface tubing 24 that is proximate rotor 16 (e.g., a region, about 3 inches long, between dotted lines 29 in FIG. 1).

According to aspects of the disclosure, treatment 28 is a layer of a material that differs from the material of tubular core 26 (e.g., it is other than Tygon® ND-100-65). For example, treatment 28 can be a layer of a nitrile formed about tubular core 26, for example by dip coating or by otherwise securing a layer of a nitrile rubber to tubular core 26. FIG. 4 depicts a representative electrogram signal resulting from the use of a nitrile layer for treatment 28.

Desirably, the electrogram signal of FIG. 4 is largely free of noise signals. The inventors believe that this advantage can be attributed, at least in part, to nitrile rubber (+3) being closer to 0 on the triboelectric scale than is the material of tubular core 26 (often around −70).

In still further aspects of the disclosure, treatment 28 is a layer of a cotton fiber. Cotton fiber (+5) is also closer to 0 on the triboelectric scale than is the material of tubular core 26 (often around −70). FIG. 5 depicts a representative electrogram signal resulting from the use of a cotton fiber layer for treatment 28.

In other aspects of the disclosure, treatment 28 is a layer of heat shrink material, such as an acrylated olefin. FIG. 6 depicts a representative electrogram signal resulting from the use of an acrylated olefin heat shrink material for treatment 28.

Treatment 28 can also be co-extruded with tubular core 26. As those of ordinary skill in the art will appreciate, co-extrusion is a process by which two or more materials are pressed through the same die to produce a single piece. Thus, treatment 28 can be a different non-DEHP material from tubular core 26, such as Tygon® E-LFL tubing, also from Saint-Gobain Performance Plastics. It is contemplated that tubular core 26 provides structural support to facilitate irrigant delivery, while treatment 28 provides an improvement in the noise signal relative to tubular core 26 alone, such as shown in the representative electrogram signal of FIG. 7, which depicts a representative electrogram signal resulting from the use of a co-extruded Tygon® E-LFL layer for treatment 28.

In other embodiments, the co-extruded material in treatment 28 includes an ESD-preventing additive mixed with a non-DEHP material, which non-DEHP material can be the same or different as the non-DEHP material of tubular core 26.

It is also contemplated that treatment 28 can be an anti-static spray applied to the outer surface of tubular core 26. FIG. 8A shows a representative electrogram signal resulting from the use of a general-purpose staticide for treatment 28, while FIG. 8B shows a representative electrogram signal resulting from the use of a licron crystal staticide for treatment 28.

Although several embodiments of this invention have been described above with a certain degree of particularity, those skilled in the art could make numerous alterations to the disclosed embodiments without departing from the spirit or scope of this invention.

For example, the teachings of the instant disclosure can be combined with those in United States patent application publication no. 2012/0165735, which is hereby incorporated by reference as though fully set forth herein, to achieve still further improvements in the minimization and/or elimination of noise signals.

As another example, although FIG. 3 depicts treatment 28 and layer 30 as two discrete layers, they could also be implemented as a single layer that incorporates both materials, such as by impregnating or blending the noise-reducing material of treatment 28 with the abrasion-resistant material of layer 30. That is, in embodiments of the disclosure, the protective material (e.g., wax, zinc stearate, erucamide, or the like) can be blended with the material of treatment 28 (e.g., nitrile).

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A pump interface tubing for use in a peristaltic pump, the pump interface tubing comprising:
a tubular core having an outer surface; and
a treatment on the outer surface of the tubular core, wherein the treatment reduces static charge buildup on the tubular core during operation of the peristaltic pump.

2. The pump interface tubing according to claim 1, wherein the tubular core comprises a first material and wherein the treatment comprises a layer of a second material different from the first material formed about the tubular core.

3. The pump interface tubing according to claim 2, wherein the second material is closer to zero on the triboelectric scale than the first material.

4. The pump interface tubing according to claim 2, wherein the second material comprises a nitrile.

5. The pump interface tubing according to claim 2, wherein the second material comprises a heat shrink material.

6. The pump interface tubing according to claim 2, wherein the second material comprises a cotton fiber.

7. The pump interface tubing according to claim 2, wherein the second material comprises an ESD-preventing additive.

8. The pump interface tubing according to claim 1, wherein the treatment comprises an anti-static spray treatment.

9. The pump interface tubing according to claim 1, wherein the treatment further resists abrasion of the tubular core during operation of the peristaltic pump.

10. The pump interface tubing according to claim 1, further comprising:
a first key at a first end of the pump interface tubing; and
a second key at a second end of the pump interface tubing,
wherein the first key and the second key are adapted to interconnect the pump interface tubing with an irrigation tube.

11. The pump interface tubing according to claim 1, wherein the treatment extends along less than an entire length of the outer surface of the tubular core.

12. The pump interface tubing according to claim 1, wherein the tubular core comprises a first layer of a material and wherein the treatment comprises a second layer of the material mixed with an ESD-preventing additive formed about the first layer of the material.

13. An irrigation system for use with an irrigated electrophysiology catheter, comprising:
a peristaltic pump; and
a pump interface tubing comprising:
a tubular core having an outer surface; and
a treatment on the outer surface of the tubular core, wherein the treatment reduces a noise signal resulting from operation of the peristaltic pump.

14. The irrigation system according to claim 13, wherein the treatment reduces static buildup on the pump interface tubing resulting from operation of the peristaltic pump.

15. The irrigation system according to claim 13, wherein the treatment comprises an anti-static layer about the tubular core.

16. The irrigation system according to claim 15, wherein the anti-static layer comprises a nitrile layer.

17. The irrigation system according to claim 15, wherein the anti-static layer comprises a heat shrink layer.

18. The irrigation system according to claim 15, wherein the anti-static layer comprises a cotton fiber layer.

19. The irrigation system according to claim 15, wherein the anti-static layer comprises an anti-static spray layer.

20. A method of manufacturing a reduced-noise pump interface tubing for use in a peristaltic pump, the method comprising forming a noise-reduction layer about a tubular core.

21. The method according to claim 20, wherein forming a noise-reduction layer about a tubular core comprises forming a nitrile layer about the tubular core.

22. The method according to claim 21, wherein forming a nitrile layer about the tubular core comprises dip-coating the nitrile layer about the tubular core.

23. The method according to claim 20, wherein forming a noise-reduction layer about a tubular core comprises co-extruding the tubular core from a first material and the noise-reduction from a second material different from the first material.

24. The method according to claim 20, wherein forming a noise-reduction layer about a tubular core comprises spraying the tubular core with an anti-static spray.

25. The method according to claim 20, wherein forming a noise-reduction layer about a tubular core comprises forming a cotton fiber layer about the tubular core.

26. The method according to claim 20, wherein forming a noise-reduction layer about a tubular core comprises forming a heat shrink layer about the tubular core.
